(19) United States
(12) Patent Application Publication
(10) Pub. No.: US 2025/0157576 A1
(43) Pub. Date: May 15, 2025

(54) SYSTEM AND METHOD FOR GENE-ENVIRONMENT ANALYSIS

(71) Applicant: Avalo, Inc., Durham, NC (US)

(72) Inventors: Emily Bellis, Durham, NC (US); Mariano Alvarez, Durham, NC (US)

(73) Assignee: Avalo, Inc., Durham, NC (US)

(21) Appl. No.: 18/948,006

(22) Filed: Nov. 14, 2024

Related U.S. Application Data
(60) Provisional application No. 63/598,674, filed on Nov. 14, 2023.
(60) Provisional application No. 63/562,130, filed on Mar. 6, 2024.

(51) Int. Cl. G16B 20/20

(52) U.S. Cl. CPC G16B 20/20

(57) ABSTRACT

The method for gene-environment analysis can include: determining an environment-variable association model, identifying causal variables associated with environmental parameters, determining a target set of causal variable values, and evaluating an organism based on the target set of causal variable values. In variants, the method can function to identify environmentally adaptive variable values (e.g., environmentally adaptive alleles), predict an optimal set of variable values (e.g., optimal genotype) for a target environment, and/or evaluate an individual organism relative to the optimal set of variable values (e.g., for breeding, for organism selection, etc.).

### CROSS-REFERENCE TO RELATED APPLICATIONS

This application claims the benefit of U.S. Provisional Application No. 63/562,130 filed 6 Mar. 2024, and U.S. Provisional Application No. 63/598,674 filed 14 Nov. 2023, each of which is incorporated in its entirety by this reference.

This application is related to U.S. application Ser. No. 18/884,930 filed 13 Sep. 2024, which is a continuation-in-part of U.S. application Ser. No. 18/374,218 filed 28 Sep. 2023, which is a continuation of U.S. application Ser. No. 18/119,030 filed 8 Mar. 2023, which claims the benefit of U.S. Provisional Application No. 63/317,656 filed 8 Mar. 2022, U.S. Provisional Application No. 63/325,831 filed 31 Mar. 2022, U.S. Provisional Application No. 63/350,326 filed 8 Jun. 2022, and U.S. Provisional Application No. 63/350,328 filed 8 Jun. 2022, each of which is incorporated in its entirety by this reference.

### TECHNICAL FIELD

This invention relates generally to the genomic field, and more specifically to a new and useful system and method for gene-environment analysis in the genomic field.

### BRIEF DESCRIPTION OF THE FIGURES

FIG. 1 is a schematic representation of a variant of the method.

FIG. 2 depicts an example of determining casual variables.

FIGS. 3A-3D depict examples of determining an association metric.

FIG. 4 depicts an illustrative example of determining association metrics.

FIGS. 5A-5C depict illustrative examples of selecting casual variables.

FIGS. 6A and 6B depict examples of identifying causal variables and determining a variable prediction model using the causal variables.

FIG. 7A depicts a first example of determining a variable prediction model.

FIG. 7B depicts a second example of determining a variable prediction model.

FIG. 8 depicts an example of evaluating an organism by determining an offset metric.

FIG. 9A depicts an illustrative example of environmental parameter values (e.g., a map of modeled environmental suitability for rainfed lowland rice) and variable values (e.g., overlaid points represent rice landraces, colored according to genotype at a genomic position associated with high habitat suitability).

FIG. 9B depicts illustrative examples of response curves for four environmental parameters (e.g., the four most important environmental parameters determining environmental suitability in FIG. 9A).

FIG. 9C depicts an illustrative example of rice genome associations with environmental suitability, determined using an environment-variable association model.

### DETAILED DESCRIPTION

The following description of the embodiments of the invention is not intended to limit the invention to these embodiments, but rather to enable any person skilled in the art to make and use this invention.

### 1. Overview

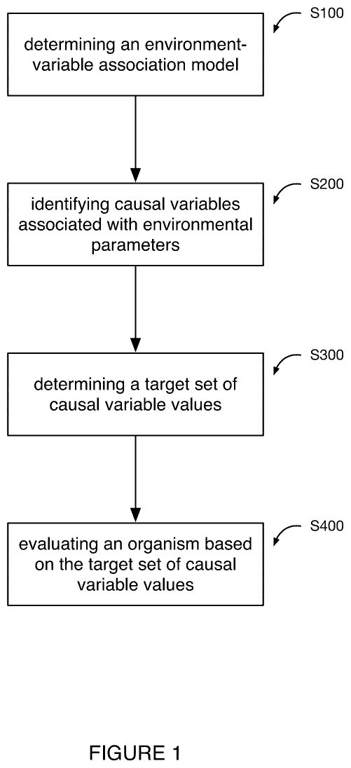

As shown in FIG. 1, the method can include: determining an environment-variable association model S100, identifying causal variables associated with environmental parameters S200, determining a target set of causal variable values S300, and evaluating an organism based on the target set of causal variable values S400. However, the method can additionally or alternatively include any other suitable steps.

In variants, the method can function to identify environmentally adaptive variable values (e.g., environmentally adaptive alleles), predict an optimal set of variable values (e.g., optimal genotype) for a target environment, and/or evaluate an individual organism relative to the optimal set of variable values (e.g., for breeding, for organism selection, etc.). However, the method can otherwise function.

### 2. Examples

In an example, the method can include: determining an environment-variable association model defining a relationship between a set of environmental parameters (response variables) and a set of variables characterizing an organism (explanatory variables); and determining causal variables (e.g., highly influential genomic positions) based on the environment-variable association model. Specific examples of variables can include: genes, loci, genomic regions, methylation, gene expression, organism identifier, spectral features, and/or any other variables characterizing an organism. In an illustrative example, the environment-variable association model can define a relationship between environmental parameters and a set of genomic positions, wherein the environment-variable association model can be trained to predict values for environmental parameters based on an allele coding value (e.g., 0, 1, or 2) at each genomic position. For example, the environment-variable association model can be trained using: known genotypes at each of the set of genomic positions for a set of crop landraces, and corresponding environmental data for the geographic locations where the crop landraces are found. The environment-variable association model can then be used to select highly influential genomic positions based on an association metric for each genomic position.

In this example, a variable prediction model can then be trained to predict values for the causal variables (e.g., predict allele coding values) given a set of environmental parameter values. For example, the variable prediction model can be trained using known environmental parameter values for a set of training organisms as the training input, and using known variable values (e.g., genotypes) for the set of training organisms as the training target. In a specific example, the training input can include: remote imagery rasters of a geographic location of a training organism, values for individual environmental parameters (e.g., temperature, rainfall, etc.) of the geographic location of the training organism, and/or any other environmental parameter values. In a first specific example, the variable prediction model can include a machine learning model. In a second specific example, the variable prediction model can include a gradient forest model. The trained variable prediction model can be used to predict a target set of causal variable values (e.g., optimal set of allele coding values for the causal genes) for a target environment defined by a target set of environmental parameter values.

In this example, a new, candidate organism characterized by a candidate set of causal variable values (e.g., candidate set of allele coding values) can optionally be evaluated by calculating a genomic offset between the candidate set of causal variable values and the target set of causal variable values. The calculated genomic offset can optionally be used for selecting an organism for breeding (e.g., to produce an organism that will exhibit elevated fitness in the target environment).

### 3. Technical Advantages

Variants of the technology can confer one or more advantages over conventional technologies.

Plant breeding for fitness in a new environment, such as a predicted future environment or a new region, has historically been extremely difficult because simulating a new environment (e.g., in a greenhouse) and evaluating the fitness of each (adult) plant generation in the new environment is inaccurate, costly, and time intensive. Additionally, the underlying relationship between genetics and environmental fitness is not well understood. Similarly, computational approaches are largely ineffective due to the immense number of potential influential variables—such as alleles, expressed genes, environmental variables, and DNA methylation—that can be permuted; the large dimensionality of the problem makes predictive gene-environment modelling extremely difficult.

First, variants of the technology can reduce the dimensionality of the search space by identifying causal variables. In examples, causal variables (e.g., causal genomic variables) can be those variables associated with positive and/or negative variable-environment association metrics. For example, the method can include generating a variable prediction model (predicting the optimal genotype for a given environment) using only the causal variables. Because the causal variables represent only a subset of all potential variables, the variable prediction model can better and more efficiently model environment-variable relationships. This reduced dimensionality can increase computational efficiency of downstream steps, including training the variable prediction model and predicting the target causal variable values.

Second, variants of the technology can predict how a plant will perform in a target environment using information that can be observed at early growth stages (e.g., the seed stage). For example, variants of the method can predict fitness of the plant in the target environment based on genotype, spectroscopy measurements of the plant, and/or any other characteristics (e.g., variable values). This early prediction can enable plant selection for breeding at the early growth stages, which can increase breeding efficiency and decrease costs.

Third, introduction of climate tolerance into elite crop varieties is of increasing importance to modern breeding programs. However, stakeholders currently lack software tools and modeling frameworks to rapidly introduce climate tolerance into elite germplasm alongside other key traits targeted by breeders. For most public and private breeding programs, climate suitability is introduced indirectly, through field evaluation of varieties that have already undergone many rounds of selection for other traits. This strategy enables the ability to select for climate tolerance during the breeding process, or to select for environmental conditions varieties do not experience during the field trial (such as future environments). Additionally, since many breeders may already be working with a set of genetically restricted germplasm, alleles important for climate resilience present in diverse germplasm material, such as crop wild relatives or landraces, may be excluded. Variants of the technology can include a breeding tool that enables selection for improved environmental suitability during the breeding process, using genomic selection for locally adaptive alleles.

However, further advantages can be provided by the system and method disclosed herein.

### 4. Method

As shown in FIG. 1, the method can include: determining an environment-variable association model S100, identifying causal variables associated with environmental parameters S200, determining a target set of causal variable values S300, and evaluating an organism based on the target set of causal variable values S400. However, the method can additionally or alternatively include any other suitable steps.

All or portions of the method can be performed once (e.g., for an individual organism, for a target environment, for a target set of environmental parameter values, for a species, for a variable, for an environmental parameter, etc.), multiple times, iteratively (e.g., for each of a set of organisms, for each of a set of target environments, for each environmental parameter in a set, for each variable in a set, etc.), in real time (e.g., responsive to a request), concurrently, asynchronously, periodically, and/or at any other suitable time. All or portions of the method can be performed automatically, manually, semi-automatically, and/or otherwise performed.

All or portions of the method can be performed using a computing system, using a database (e.g., a system database, a third-party database, etc.), using a genomic sequencer, using assay tools, using measurement systems, using chemical instruments, using a user interface, by a user, and/or by any other suitable system. The computing system can include one or more: CPUs, GPUs, TPUs, custom FPGA/ASICS, microprocessors, servers, cloud computing, and/or any other suitable components. The computing system can be local, remote (e.g., cloud computing server, etc.), distributed, and/or otherwise arranged relative to any other system or module.

Measurement systems can include imaging sensors, environmental sensors (e.g., temperature sensors), chemical sensors, sequencing systems (e.g., sensors used for whole-genome sequencing, etc.), and/or other sensors. Measurements (e.g., data) acquired by the measurement systems can include: images (e.g., spectral data, photographs, etc.), environmental data (e.g., temperature, precipitation, environmental images, and/or any other observed values for environmental parameters), genomic data (e.g., DNA sequences, RNA sequences, epigenetic information, genotypes, and/or any other observed values for genomic variables), and/or any other measurements. In specific examples, imaging sensors can include spectrometers (e.g., infrared and/or near infrared spectrometers, ultraviolet spectrometers, visible light spectrometers, any other wavelength, etc.), lidar sensors, cameras (e.g., multispectral camera), and/or any other imaging sensor. Measurements acquired by the imaging sensors can include measurements of an individual organism (e.g., to determine observed variable values) and/or measurements of a geographic region (e.g., to determine environmental parameter values). In a specific example, measurements of an individual organism can include spectral data (e.g., spectra of a seed, spectra of a leaf, etc.). In a specific example, measurements of a geographic region can include remote measurements (e.g., aerial imagery, satellite imagery, balloon imagery, drone imagery, etc.), local or on-site measurements, and/or any other measurements of the geographic region.

Measurements of an organism can be acquired during one or more stages of organism (e.g., plant) development. In a first example, the data for an organism can be acquired when the organism is a seed. In a second example, the data for an organism can be acquired during and/or after organism development (e.g., after harvesting). In a third example, a first set of data for an organism can be acquired when the organism is a seed and a second set of data for the organism can be acquired during and/or after development.

The method can include or use one or more models, including an environmental suitability model, an environment-variable association model, a variable-variable association model, a variable prediction model, an offset model, and/or any other model.

The models can use classical or traditional approaches, machine learning approaches, and/or be otherwise configured. The models can include regression (e.g., linear regression, non-linear regression, logistic regression, etc.), decision tree (e.g., random forest, gradient forest, classification and regression tree, iterative dichotomiser 3, C4.5, chi-squared automatic interaction detection, decision stump, multivariate adaptive regression splines, gradient boosting machines, etc.), clustering methods (e.g., k-means clustering, hierarchical clustering, expectation maximization, etc.), association rules, dimensionality reduction (e.g., principal component analysis, t-distributed stochastic neighbor embedding, linear discriminant analysis, partial lest squares regression, Sammon mapping, multidimensional scaling, projection pursuit, etc.), neural networks (e.g., GNN, CNN, DNN, CAN, LSTM, RNN, encoders, decoders, transformers, etc.), ensemble methods (e.g., boosting, bootstrapped aggregation, stacked generalization, gradient boosting machine method, random forest method, etc.), optimization methods (e.g., Bayesian optimization, convex optimization, non-convex optimization, multi-objective optimization, etc.), classification, rules, heuristics, equations (e.g., weighted equations, etc.), selection (e.g., from a library), lookups, regularization methods (e.g., ridge regression, Lasso regression, etc.), Bayesian methods (e.g., Naive Bayes, Markov, hidden Markov models, etc.), instance-based methods (e.g., nearest neighbor), kernel methods, encoders (e.g., autoencoders), support vectors, statistical methods (e.g., probability), comparison methods (e.g., matching, distance metrics, thresholds, etc.), deterministics, genetic programs, foundation models, generative models, process models, biological models, and/or any other suitable model. Models can be low-dimension models, high-dimension models, and/or otherwise configured.

Models can be trained, learned, fit, predetermined, and/or can be otherwise determined. The models can be trained or learned using: supervised learning (e.g., using logistic regression, using back propagation neural networks, using random forests, decision trees, etc.), unsupervised learning, self-supervised learning, semi-supervised learning (e.g., positive-unlabeled learning), reinforcement learning, transfer learning, Bayesian optimization, fitting, interpolation and/or approximation (e.g., using gaussian processes), backpropagation, and/or otherwise generated. The models can be learned or trained on: labeled data (e.g., data labeled with the target label), unlabeled data, positive training sets (e.g., a set of data with true positive labels), negative training sets (e.g., a set of data with true negative labels), and/or any other suitable set of data. Training datasets can optionally be upsampled, downsampled, debiased, and/or otherwise preprocessed. Any model can optionally be updated and/or retrained based on newly received, up-to-date measurements; past measurements recorded during the operating session; historic measurements recorded during past operating sessions; or be updated based on any other suitable data.

The method can be used with one or more variables. Variables are preferably characteristics associated with an organism, but can be otherwise defined. Examples of variables include: genomic variables (e.g., genomic components, gene expression, etc.), protein variables (e.g., protein expression), methylation variables (e.g., which DNA positions are methylated, overall amount of methylation, etc.), transcriptome variables, microbial variables (e.g., for microbes associated with the organism), biologic variables (e.g., biological markers), environmental parameters, phenotype variables, measurements (e.g., spectra, images, etc.), features thereof (e.g., features extracted from a sequence, features extracted from a measurement, etc.), a combination thereof, and/or any other characteristic associated with an organism. Genomic components are preferably basic units shared across all organisms of a population (e.g., a species), but can alternatively be otherwise defined. Examples of genomic components include: a gene, a gene group, a locus (e.g., DNA or RNA) and/or other genomic position, a gene region, RNA region, RNA transcript identifier, k-mer, and/or any other genomic component. The set of variables preferably includes all variables, but can alternatively include a subset of the variables (e.g., the variables that can be controlled, etc.), and/or be otherwise defined. For example, the variable set can include: all possible loci, loci of interest, all possible genes (e.g., all genes of one or more organisms in the population), expressible protein, environmental variables, genes of interest, all genomic regions (e.g., nonoverlapping or overlapping), genomic regions of interest, all methylated locations, methylated locations of interest, DNA and/or RNA sequences, all environmental parameters, environmental parameters of interest, and/or any other variables.

Variable values (e.g., a value for a given variable) can be qualitative, quantitative, relative, discrete, continuous, a classification, numeric, binary, and/or be otherwise characterized. Variable values can be measured, extracted from measurements, retrieved, determined based on other variable values, determined using a model, synthetically determined, simulated, predicted, predetermined, randomly determined, manually determined, and/or otherwise determined. Variable values can include observed values (e.g., measured, extracted from measurements, predicted from measurements, etc.) and/or test values (e.g., synthetic values for a variable being tested; determined by removing information from the corresponding variable; etc.). In a first example, variable values can include genomic component values, including: genotypes, DNA and/or RNA sequences, single nucleotide polymorphisms (SNPs), k-mers, k-mer counts, RNA counts, allele coding, presence/absence of a genomic component (e.g., of a particular gene sequence), evolutionary history, heredity history, DNA fragmentation, and/or any other genetic and/or cellular information. In a specific example, a genomic component value can be a numerical value representing the genotype (e.g., an allele coding) for an organism at a gene locus associated with the variable. In examples, an allele coding can include a 0, 1, or 2 value (e.g., determined based on allele frequency in the population) and/or any other values (e.g., 0-9 values when more than two copies of an allele are present in the population). In a second example, variable values can include spectral feature values. In a specific example, feature values can be extracted from a measured spectrum (e.g., NIR spectrum). However, variables and/or variable values can be otherwise defined.

The method can be used with one or more environmental parameters. Examples of environmental parameters include: temperature; growing degree days, pressure; light; humidity; rainfall; growing medium (e.g., soil) composition (e.g., nutrient composition, pH, moisture, etc.), growing medium quality; water availability; land grade; concentration and/or distribution of macronutrients and/or micronutrients (e.g., nitrogen, phosphorous, etc.); growing duration; treatment frequency; environmental suitability; features thereof; aggregates thereof (e.g., average, median, etc.); a temporal characteristic thereof; a geographic characteristic thereof; a combination thereof; and/or any other characteristic of an organism's environment.

Environmental parameter values (e.g., values for environmental parameters) can be qualitative, quantitative, relative, discrete, continuous, a classification, numeric, binary, and/or be otherwise characterized. Environmental parameter values can be measured, extracted from measurements, calculated, retrieved from a database (e.g., a third-party database), determined using a model, synthetically determined, predetermined, randomly determined, manually determined, and/or otherwise determined. Environmental parameter values can include observed values (e.g., measured, extracted from measurements, predicted from measurements, etc.), and/or test values. Environmental parameter values can optionally be associated with a time (e.g., when the corresponding measurement was collected) and/or a geographic region (e.g., where the corresponding measurement was collected). Each environmental parameter can optionally correspond to a set of environmental parameter values. In a first example, the set of environmental parameter values for a given environmental parameter can be a single value (e.g., average temperature, average rainfall, an environmental suitability metric, etc.). In a second example, the set of environmental parameter values for a given environmental parameter can be a vector of values. In a specific example, the set of environmental parameter values can be a timeseries of values (e.g., temperature timeseries, rainfall timeseries, etc.). In a third example, the set of environmental parameter values for a given environmental parameter can be one or more rasters (e.g., a timeseries of rasters). The one or more rasters can include: discrete (e.g., thematic) raster data (e.g., land-use data, soil data, wildfire risk data, etc.), continuous raster data (e.g., temperature data, precipitation data, elevation data, atmospheric carbon dioxide data, spectral data, satellite images, etc.), and/or any other raster data.

In a first embodiment, environmental parameter values can include environmental data. For example, the environmental data can include: number of growing degree days, temperature values, pressure values, growing medium (e.g., soil) composition values (e.g., nutrient concentration, pH, moisture level, etc.), humidity level, ultraviolet light level, rainfall level, rasters, other measurements, temporal values thereof, and/or other measures of environmental parameters. In a specific example, environmental parameter values for an organism can be determined based on the geographic region where the organism is located. In an illustrative example, a database (e.g., germplasm database) can include a location associated with each landrace in a set of crop landraces (e.g., where the gene sequence and/or other observed variable values for the crop landraces can be included in the database and/or otherwise determined); the location can then be used to determine the environmental parameter values for the respective landrace.

In a second embodiment, environmental parameter values can include an environmental suitability metric representing the suitability of a given habitat for the organism. The environmental suitability metric can be qualitative, quantitative, relative, discrete, continuous (e.g., value between 0 and 1), a classification, numeric, binary, and/or be otherwise characterized. In a specific example, an environmental suitability metric can be output by an environmental suitability model. Inputs to the environmental suitability model can include environmental parameter values (e.g., a vector of environmental parameter values) and/or any other suitable inputs. A specific example of the environmental suitability model can include MaxEnt and/or any other presence-only modeling method. In a first example, training data for training the environmental suitability model can include known locations of crop landraces and/or other organisms (e.g., and associated environmental parameter values for the locations), environmental parameter values for locations where the organism(s) are not found (e.g., pseudoabsences), yield data for each of a set of locations (e.g., and associated environmental parameter values for the locations), and/or any other training data.

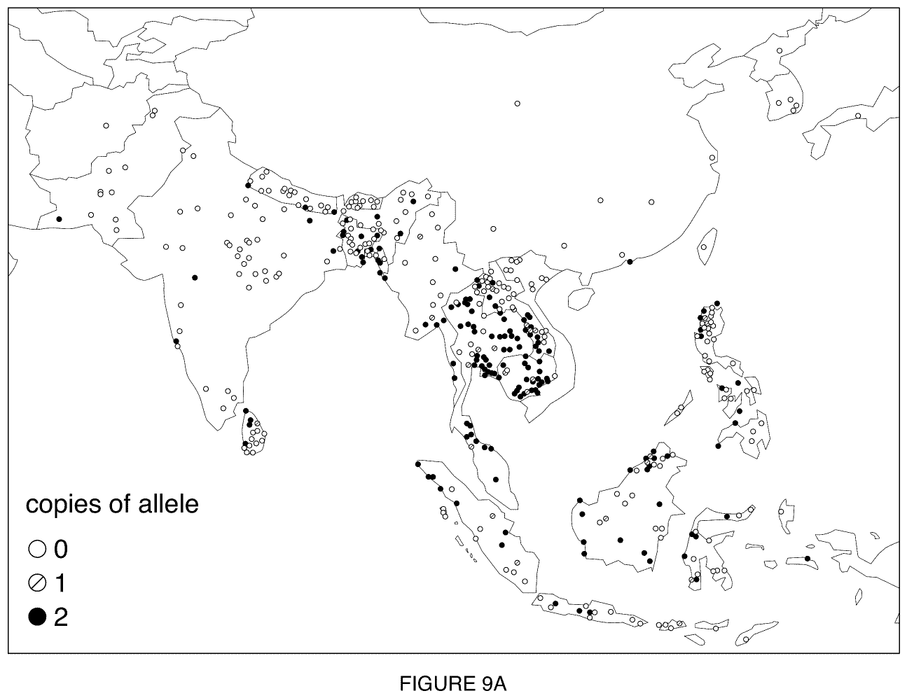

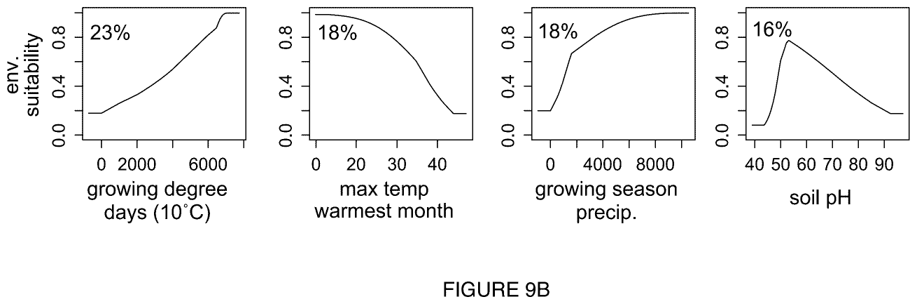

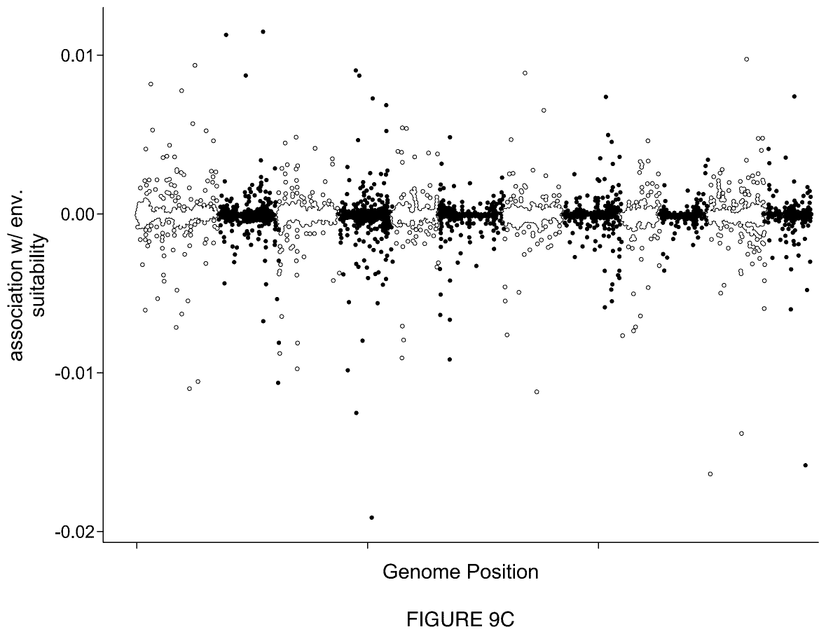

The method can optionally include identifying a subset of environmental parameters (e.g., the most important environmental parameters determining environmental suitability). An example is shown in FIG. 9B. The subset of environmental parameters can be determined using the environmental suitability model (e.g., causal environmental parameters that influence environmental suitability), using the environmental-variable model, predetermined, manually determined, and/or otherwise determined. In a specific example, the environmental suitability model can be used to determine a feature importance score for each environmental parameter, wherein the high-importance environmental parameters are selected as the subset of environmental parameters. The subset of environmental parameters can be used as the set of environmental parameters in all or parts of the method.

Each variable and/or environmental parameter can optionally be a vector or matrix including the values (e.g., observed values and/or test values) for the respective variable or environmental parameter for each of a set of organisms (e.g., ordered set of organisms). In a first example, a genomic component variable can be a vector of genomic component values (e.g., representing genotypes corresponding to the genomic component, representing k-mers corresponding to the genomic component, etc.) with one genomic component value in the vector for each organism in the population. In a second example, an environmental parameter can be a vector of environmental parameter values with one environmental parameter value for each organism in the population. In a specific example, environmental parameter values can include individual numerical values (e.g., average temperature), wherein an environmental parameter can be a vector with one numerical value for each organism in the population. In a third example, environmental parameter values can include raster images, wherein an environmental parameter can include one or more raster images for each organism in the population (e.g., where the one or more raster images are associated with the target location and/or the collection location of the respective organism).

Values for variables and/or values for environmental parameters can optionally be transformed. Transformed variable values and/or transformed environmental parameter values can optionally be treated as variable values and/or environmental parameter values, respectively, in all or parts of the method. For example, variable values can be transformed (e.g., embedded) to a reduced dimension space (e.g., latent space), wherein the transformed variable values can be treated as variable values in all or parts of the method.

However, the variables, environmental parameters, and/or values thereof can be otherwise represented.

### 4.1. Determining an Environment-Variable Association Model S100.

Determining an environment-variable association model S100 functions to determine a model defining a relationship between the set of environmental parameters and the set of variables (e.g., genomic variables). For example, the environment-variable association model can predict environmental parameter values given variable values.

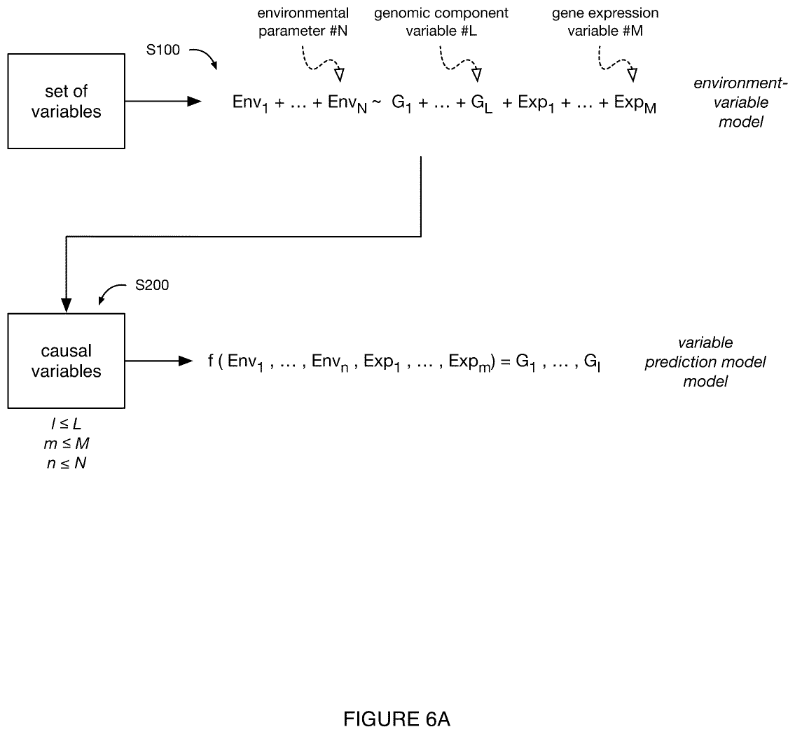

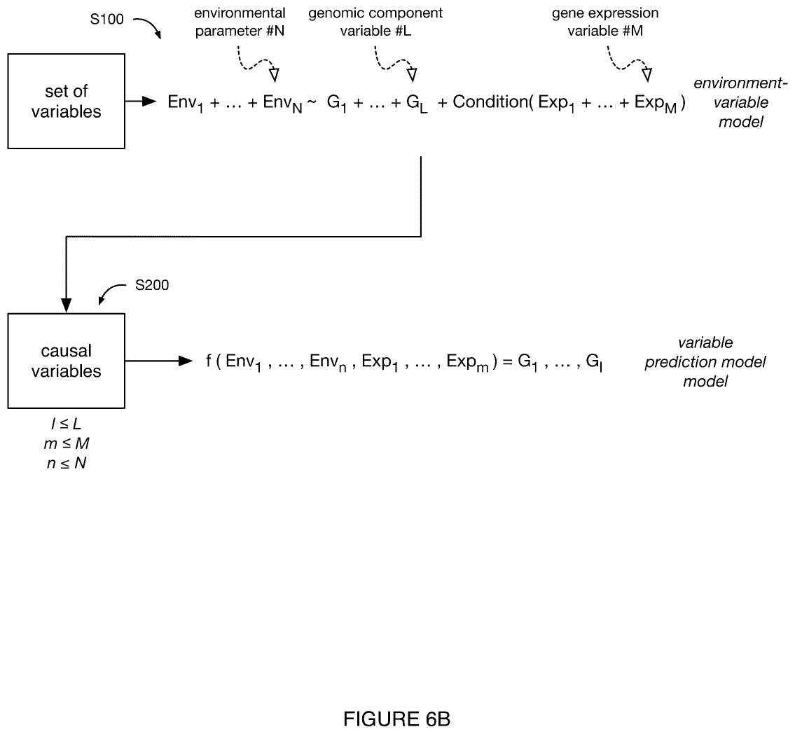

Determining the environment-variable association model can include training a model to predict one or more environmental parameter values corresponding to an organism's environment given the organism's variable values (e.g., a value for each of the set of variables). Examples are shown in FIG. 6A and FIG. 6B. The environment-variable association model can be: for a specific environmental parameter (e.g., environmental suitability), for a set of environmental parameters (e.g., temperature, rainfall, etc.), and/or any other suitable combination of environmental parameters. The set of environmental parameters used in the environment-variable association model preferably does not include environmental parameters corresponding to rasters, but can alternatively include environmental parameters corresponding to rasters. The environment-variable association model can optionally predict a value for one or more environmental parameters based on values for a set of variables conditioned on a set of covariates, wherein the set of covariates can include other variables (e.g., gene expression variables, DNA methylation variables, etc.); an example is shown in FIG. 6B. The environment-variable association model can optionally account for batch effects (naturally occurring batch effects). In a specific example, the environment-variable association model can predict a value for one or more environmental parameters based on values for a set of variables conditioned on population structure (e.g., using a subspecies identifier as a covariate). The environment-variable association model preferably does not model inter-variable interactions, but alternatively can model inter-variable interactions.

The environment-variable association model can be determined (e.g., trained) using: environmental parameter values, observed variable values, test variable values, and/or any other information for one or more training organisms. In a first variant, the environment-variable association model is a neural network trained using variable values (e.g., genotypes) for a set of training organisms as the training input, and using observed environmental parameter values for the set of training organisms as the training target. In a second variant, the environment-variable association model is a regression (e.g., linear regression, nonlinear regression, multivariate regression, etc.) fit to the observed environmental parameter values and the variable values, wherein the environmental parameters are treated as the dependent variables and the variables are treated as the independent variables. The variable values used for training the environment-variable association model can include only observed variable values, only test variable values, or a combination of observed variable values and test variable values. However, the environment-variable association model can be otherwise determined.

In an example, the environment-variable association model can be determined using methods disclosed in U.S. application Ser. No. 18/884,930 filed 13 Sep. 2024, U.S. application Ser. No. 18/119,048 filed 8 Mar. 2023, and/or U.S. application Ser. No. 18/374,218 filed 28 Sep. 2023, each of which is incorporated in its entirety by this reference. In a specific example, methods for determining a phenotype-variable association model can be used for determining the environment-variable association model.

However, the environment-variable association model can be otherwise determined.

4.2. Identifying Causal Variables Associated with Environmental Variables S200.

Identifying causal variables associated with environmental variables S200 functions to reduce the variable dimensionality, identify environmentally adaptive variables, and/or identify influential variables associated with high habitat suitability (e.g., organism fitness for a given environment). The causal variables (e.g., causal genomic variables) can be identified (e.g., selected) for: a set of environmental parameters, a set of environmental parameter values, a set of organisms (e.g., a species, subspecies, etc.), and/or otherwise identified.

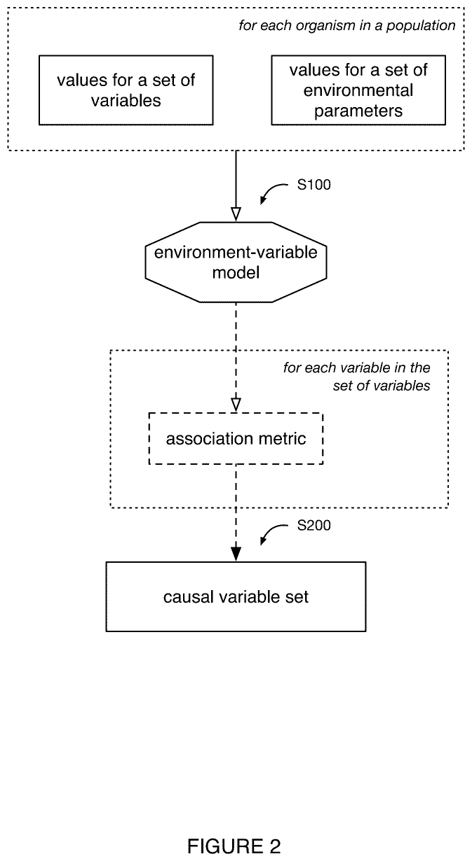

The causal variables can be identified using the environment-variable association model, using a database, manually, predetermined, randomly, and/or otherwise selected. Identifying the causal variables can include: determining an association metric for each variable in a plurality of variables based on the environment-variable association model; and selecting the causal variables from the plurality of variables based on the respective association metric for each variable. An example is shown in FIG. 2.

Determining an association metric for each variable based on the environment-variable association model can function to extract information on the relationship between each variable and one or more environmental parameters. For example, an association metric for a variable can be a measure of that variable's importance in the environment-variable association model.

The association metric for a variable can be a comparison between model metrics (e.g., a loss metric) for an observed model (an observed environment-variable association model) and a test model (a test environment-variable association model), trained on observed values and test values for the variable, respectively. Additionally or alternatively, the association metric for a variable can be: a classification (e.g., causal or non-causal variable), a model metric value, a measure of association between the variable and one or more environmental parameters (e.g., determined based on a coefficient of the variable in the environment-variable association model), and/or otherwise defined. Examples of model metrics include: the variable weight (e.g., a coefficient in the model), the model's environmental parameter value prediction, the model's loss, the model's variance (e.g., coefficient of determination), and/or any other value determined based on the environment-variable association model. Association metrics for different variables can be independently determined or, alternatively, can be concurrently determined. Multiple variables can optionally be associated with the same association metric.

Determining an association metric for a variable can optionally include determining a test variable (e.g., a vector of one or more test values for a variable). For example, an observed variable (e.g., including observed values for each training organism) and/or a test variable (e.g., including test values for each training organism) can be determined for a variable of interest. Determining the test variable can function to remove the variable's information. For example, determining the test variable can remove the information from the corresponding observed variable while maintaining a suitable variable form and/or distribution such that an original observed variable can be exchanged with its corresponding test variable. The test variable preferably has the same or substantially the same distribution (e.g., statistical distribution) as the corresponding observed variable, but alternatively can have a different distribution. In a specific example, the set of causal variables can be selected from a set of variables using an observed value and a test value for each variable in the set of variables.

The test variable can be generated using a variable-variable association model, be randomly determined, be perturbed, be manually determined, and/or be otherwise determined. In a first variant, determining a test variable for a corresponding observed variable can include replacing the observed variable values with null values. In a second variant, determining a test variable can include randomly generating values to replace the observed variable values. In a third variant, determining a test variable can include adding noise to the observed variable values. In a fourth variant, determining a test variable can include determining a distribution of observed variable values based on the corresponding observed variable, and generating test variable values to match the distribution (e.g., a genotype distribution). For example, the distribution can be modeled (e.g., as a gaussian distribution), wherein test variable values can be randomly selected from the modeled distribution. In a fifth variant, the test variable can be determined using a process model (e.g., representing how the variable values are generated). For example, the process model can be a forward-in-time evolution model. In a sixth variant, determining a test variable can include determining (e.g., training) a variable-variable association model, and determining the test variable using the variable-variable association model.

Inputs to the variable-variable association model can include: variable values (e.g., observed variables including observed variable values), an optional randomization parameter (e.g., a parameter that can introduce randomness in the model pre- or post-training), and/or any other suitable inputs. For example, observed variable inputs can include (only) the observed variables corresponding to a variable window. Outputs from the variable-variable association model can include: variable values (e.g., a test variable including test variable values), and/or any other suitable outputs. For example, test variable outputs can include a single test variable (associated with one or more observed variables) or multiple test variables (e.g., each associated with one or more observed variables). In an example, the variable-variable association model can be a model trained to predict a value for a variable of interest based on values for a subset of variables (e.g., wherein the variable of interest is not in the subset of variables). In a first example, the variable-variable association model can be or include a regression fit to observed variables, where the observed variable for the variable of interest is treated as the dependent variable and observed variables in the variable window are treated as the independent variables. In a second example, the variable-variable association model can be or include a machine learning model (e.g., an autoencoder, CNN, etc.) trained to predict test variable values based on the values for other observed variables.

The variable window can be a subset of variables, wherein the corresponding observed variables in the variable window can be used to predict a test variable for the variable of interest (e.g., wherein the variable corresponding to the test variable is not within the variable window). The variable window size and/or other parameters can be fixed or variable (e.g., based on the variable of interest). The variable window is preferably positioned relative to the variable of interest (e.g., centered about the variable of interest, offset from the variable of interest, start or end from the variable of interest, be within a threshold distance from the variable of interest, etc.), but can be otherwise positioned. The variable window can be symmetric about the variable of interest or non-symmetric. The variable window can be manually determined, determined using a model, predetermined, randomly determined, and/or otherwise determined. In a first variant, the variable window is fixed relative to the variable of interest (e.g., the variable window can be a fixed size, and positioned symmetric about the variable of interest). In a second variant, the variable window can be determined based on variable analyses (e.g., linkage disequilibrium; variable of interest location; local autocorrelation patterns; correlation strength; etc.). In a third variant, the variable window can be adaptively determined. For example, the variable window can be iteratively re-determined (e.g., using a variable window model) until one or more criteria are satisfied. The criteria can include a variable window evaluation metric criterion (e.g., the variable window model metric rises above a threshold), a number of iterations, a number of iterations without an increase in the model metric, completing a cycle through all variables in the variable set (e.g., in the variable subset), a threshold criterion, and/or any other criterion. However, the variable window can be otherwise determined.

In an example, test variables can be determined using methods disclosed in U.S. application Ser. No. 18/884,930 filed 13 Sep. 2024, U.S. application Ser. No. 18/119,048 filed 8 Mar. 2023, and/or U.S. application Ser. No. 18/374,218 filed 28 Sep. 2023, each of which is incorporated in its entirety by this reference.

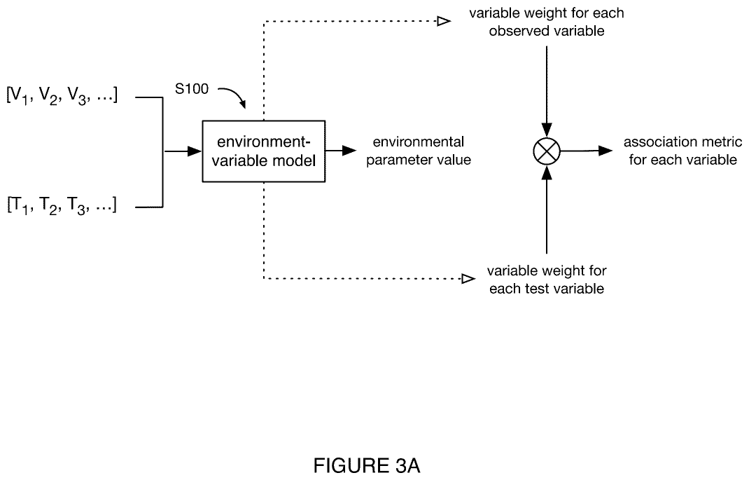

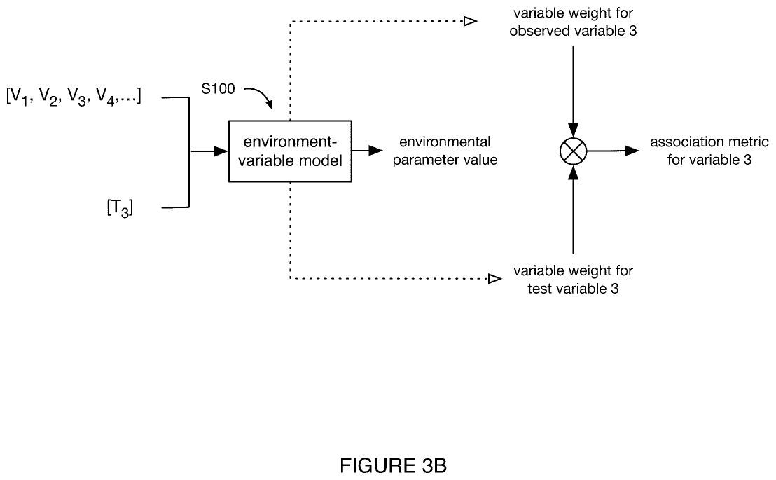

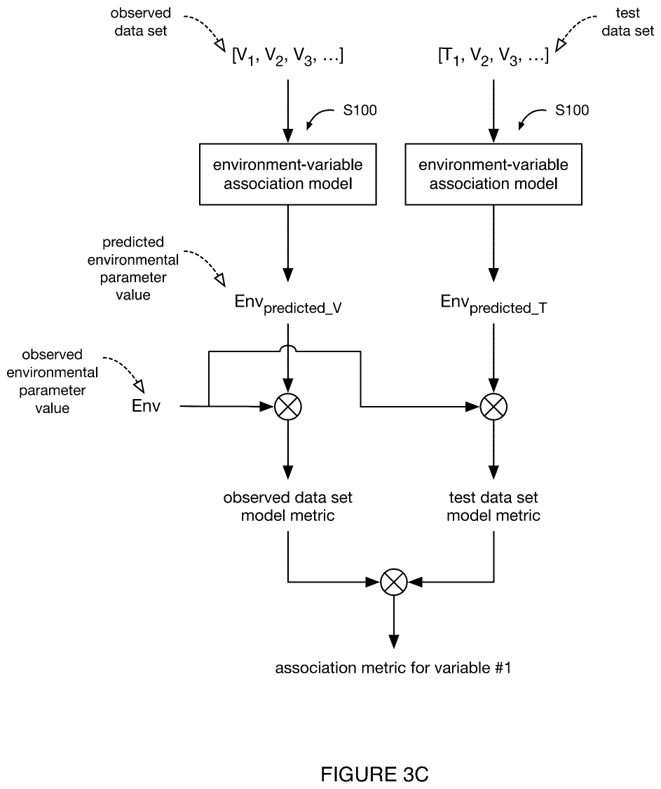

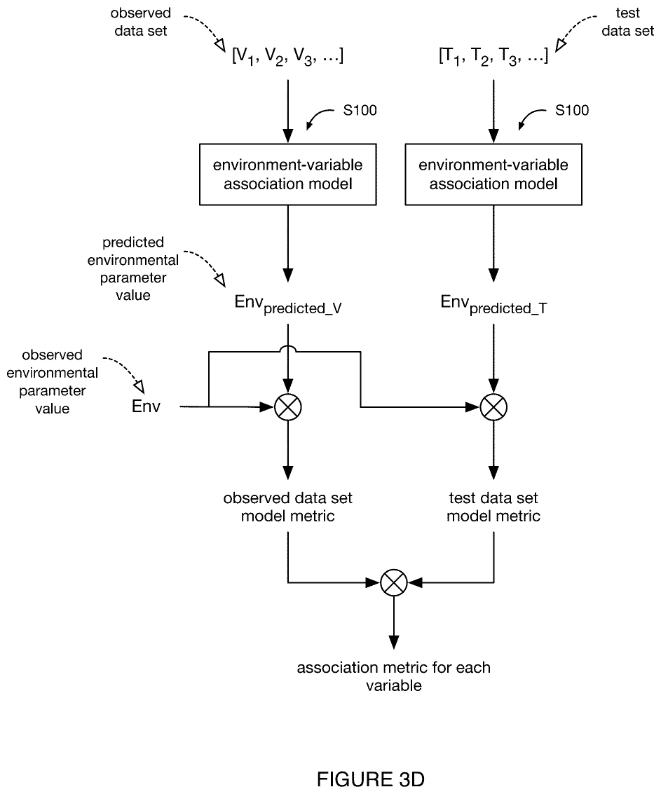

In a first variant, determining the association metric for a variable of interest can include determining a first environment-variable association model using observed values for the variable of interest (e.g., the “observed model”) and determining a second environment-variable association model using test values for the variable of interest (e.g., the “test model”). The observed model can be trained using observed values for each variable in the set of variables (including the variable of interest). In a specific example, training the observed model can include determining weights for one or more variables (e.g., for each observed variable, for the variable of interest, etc.). In a first example, the test model can be trained using test values for the variable of interest, and using observed values for the other variables in the set of variables. In a second example, the test model can be trained using test values for each variable in the set of variables. In a specific example, training the test model can include determining weights for one or more variables (e.g., for each observed variable, for each test variable, for the variable of interest, etc.). The association metric for the variable of interest can be determined based on a comparison between a model metric for the observed model to a model metric for the test model. In a first specific example, the association metric can be a difference between a model loss calculated for the observed model and a model loss calculated for the test model; examples are shown in FIG. 3C and FIG. 3D. In a second specific example, the association metric can be a comparison (e.g., difference, ratio, etc.) between a coefficient for the variable of interest in the observed model and a coefficient for the variable of interest in the test model.

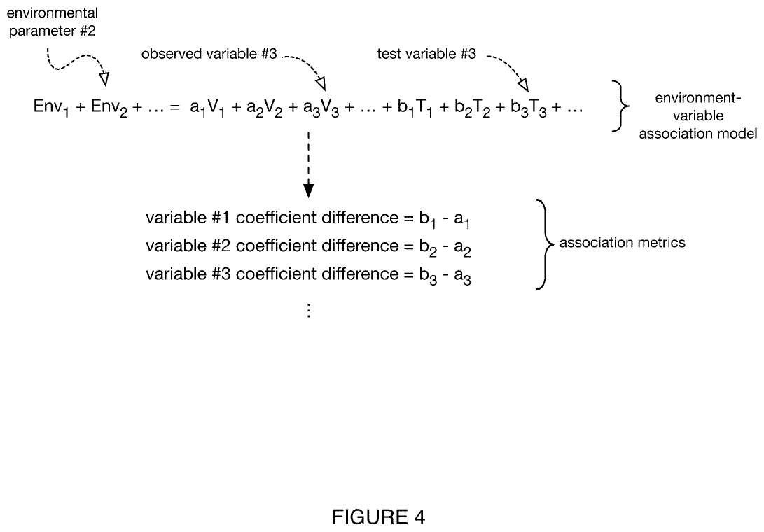

In a second variant, determining the association metric for a variable of interest can include determining a single environment-variable association model using observed values and test values for the variable of interest. An example is shown in FIG. 3A and FIG. 3B. In a first example, the environment-variable association model can be trained using observed values and test values for each variable of interest in the set of variables. In a second example, the environment-variable association model can be trained using observed values for each variable in the set of variables and using test values for the variable of interest. The association metric can be a comparison (e.g., difference, ratio, etc.) between a coefficient for the observed variable (e.g., the vector of observed values for the variable of interest) and a coefficient for the corresponding test variable (e.g., the vector of test values for the variable of interest). An example is shown in FIG. 4.

In a third variant, the association metric for a variable of interest can be determined without using test values for the variable. For example, the association metric can be a coefficient for the variable in the environment-variable association model (e.g., where test variable values are not used in the environment-variable association model).

In a fourth variant, the association metric (overall association metric) for a variable of interest can be determined using based on multiple association metrics for the variable of interest. In a first example, multiple association metrics for the variable of interest can be determined using different subsamples of variables in a subset of variables (e.g., wherein the variable of interest is not contained within the subset of variables or any subsample thereof). In a second example, variables can be grouped (e.g., clustered) into a set of groups, wherein an association metric can be determined for each group of the set of groups. In a specific example, an association metric for the variable of interest can be determined based on an association metric determined for each group associated with (e.g., containing) the variable of interest (e.g., wherein the variable of interest can be associated with multiple groups). In an example, the variables can be grouped according to a hierarchical grouping (e.g., each variable is contained in one group in each layer (e.g., tier) of the hierarchy, where the groups become progressively larger or smaller). In an example, at each layer of the hierarchy, an association metric associated with the variable of interest can be determined based on the subset of variables within the group (in the respective layer) containing the variable of interest. An overall association metric for the variable of interest can be determined based on how the association metric for the variable of interest changes across the layers of the hierarchy (e.g., if the association metric increases or decreases as the groups becomes smaller). In specific examples, the variables can be grouped using clustering methods (e.g., unsupervised clustering), domain knowledge, evolutionary history, heredity history, linkage disequilibrium, and/or any other grouping methods.

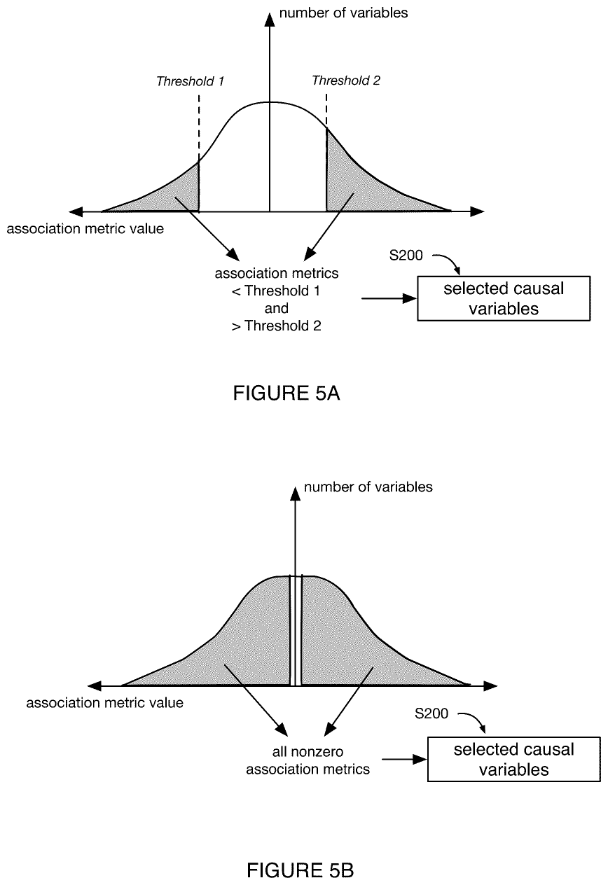

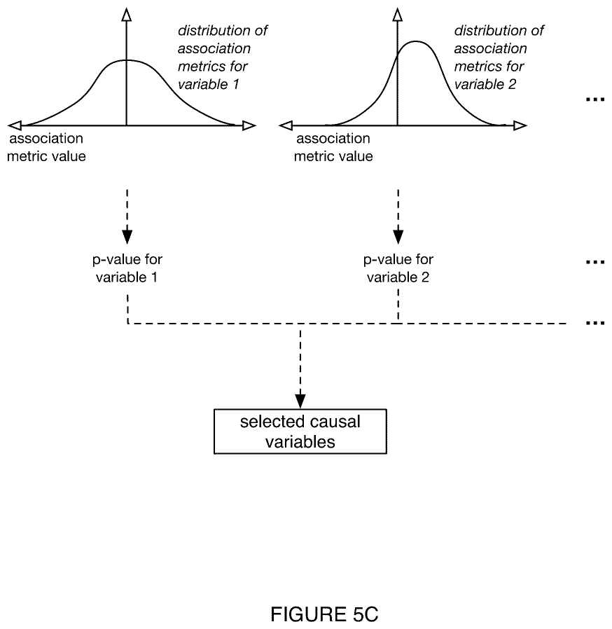

In a fifth variant, the association metric (e.g., overall association metric) for a variable of interest can be determined based on a distribution for the variable of interest. The distribution can optionally be a distribution of association metrics (e.g., a predicted distribution of association metrics, an estimated distribution of association metrics, etc.). In a first embodiment, one or more association metrics can be determined for a variable of interest (e.g., as previously described), and a distribution of association metrics can then be determined based on the one or more association metrics. For example, the distribution of association metrics for a variable of interest can be estimated based on multiple association metrics determined for the variable of interest. In a second embodiment, the distribution for a variable of interest can be determined based on the distribution of association metrics of a subset of variables. In a third embodiment, the distribution for a variable of interest can be determined based on a distribution of model metrics (e.g., coefficients) for a subset of variables (e.g., a subset of variables does not include the variable of interest). In an illustrative example, the distribution of association metrics can be the same as the distribution of model metrics for the subset of variables. In a fourth embodiment, a combination of the previous embodiments can be used. The distribution associated with a variable can be normal, non-normal, gaussian, and/or any other distribution type. The causal variables can optionally be selected based on a statistical analysis determined based on the distribution of association metrics for each variable. For example, the (overall) association metric for a variable of interest can be a statistical analysis (e.g., a p-value) determined based on the distribution of association metrics for the variable of interest. In a specific example, the p-value can be adjusted using a false discovery rate correction (e.g., a Benjamini-Hochberg procedure). An example is shown in FIG. 5C.

However, association metrics can be otherwise determined.

Selecting the causal variables from the plurality of variables based on the respective association metric for each variable can include selecting: variables with nonzero (e.g., positive and negative) association metrics, variables with association metrics above a threshold (e.g., absolute value above a threshold), variables with association metrics above a first (positive) threshold and variables with association metrics below a second (negative) threshold, a predetermined number and/or percent of variables with the largest positive association metric values, a predetermined number and/or percent of variables with the largest negative association metric values, and/or any other variable subset. An example is shown in FIG. 5A and FIG. 5B. Thresholds for causal variable selection can optionally be determined using a cost function. Additionally or alternatively, all variables of a specific type and/or classification can be selected (e.g., all environmental variables). Illustrative examples of determining and/or using causal variables (e.g., genomic positions associated with high habitat suitability) are shown in FIG. 9A and FIG. 9C.

In an example, the causal variables can be identified using methods disclosed in U.S. application Ser. No. 18/884,930 filed 13 Sep. 2024, U.S. application Ser. No. 18/119,048 filed 8 Mar. 2023, and/or U.S. application Ser. No. 18/374,218 filed 28 Sep. 2023, each of which is incorporated in its entirety by this reference. In a specific example, methods for identifying causal variables using a phenotype-variable association model can be used for identifying causal variables using the environment-variable association model.

However, the causal variables can be otherwise identified.

### 4.3. Determining a Target Set of Causal Variable Values S300.

Determining a target set of causal variable values S300 functions to predict optimal values for the causal variables (e.g., an optimal genotype) for a target environment.

The target set of causal variable values (e.g., target values for the causal variables) is preferably determined for a target environment, but can alternatively be determined for a general environment and/or otherwise determined. The target environment can optionally be defined by values for a set of environmental parameters (e.g., for a subset of high-importance environmental parameters). The set of environmental parameters can be the same or different (e.g., overlapping or nonoverlapping) set of environmental parameters as used in S100 and/or S200. In an illustrative example, the set of environmental parameters used in the environment-variable association model can include environmental suitability, while the set of environmental parameters used in the variable prediction model does not include environmental suitability. In another illustrative example, values for the set of environmental parameters used in the environment-variable association model do not include rasters, while values for the set of environmental parameters used in the variable prediction model (e.g., training values for the set of environmental parameters used to train the variable prediction model, target values for the set of environmental parameters used for determining target causal variable values, etc.) does include rasters. In a first example, the target environment can include environmental parameter values (e.g., measured, estimated, and/or predicted values) for a target time period (e.g., the upcoming year, 5 years in the future, 10 years in the future, etc.) and/or for a target region (e.g., target geographical location). In an illustrative example, the target region can be a region where the organism previously was not previously farmed. In a second example, the target environment can include environmental parameter values for a geographic region exposed to a hazard (e.g., hail, wildfire, hurricane, flooding, etc.).

The target set of causal variable values can be determined using a variable prediction model, retrieved, predetermined, randomly determined, manually determined, and/or otherwise determined.

Inputs to the variable prediction model can include values for a set of environmental parameters (e.g., environmental parameter values for the target environment) and/or any other suitable inputs. The set of environmental parameters can be the same set of environmental parameters used in the environment-variable association model or a different, second set of environmental parameters. The variable prediction model preferably accepts multiple inputs (e.g., values for multiple environmental parameters). The variable prediction model preferably accepts multiple input types (e.g., single values, vectors, rasters, etc.). Outputs from to the variable prediction model can include variable values (e.g., the target set of causal variable values) and/or any other suitable outputs. In an example, the set of causal variables includes genomic positions, wherein the target values for the set of causal variables includes genotypes at the genomic positions. In an illustrative example, the variable prediction model can output an optimal set of alleles (e.g., optimal set of genotypes for causal genes) that are predicted to optimize organism fitness when exposed to the target set of environmental parameter values.

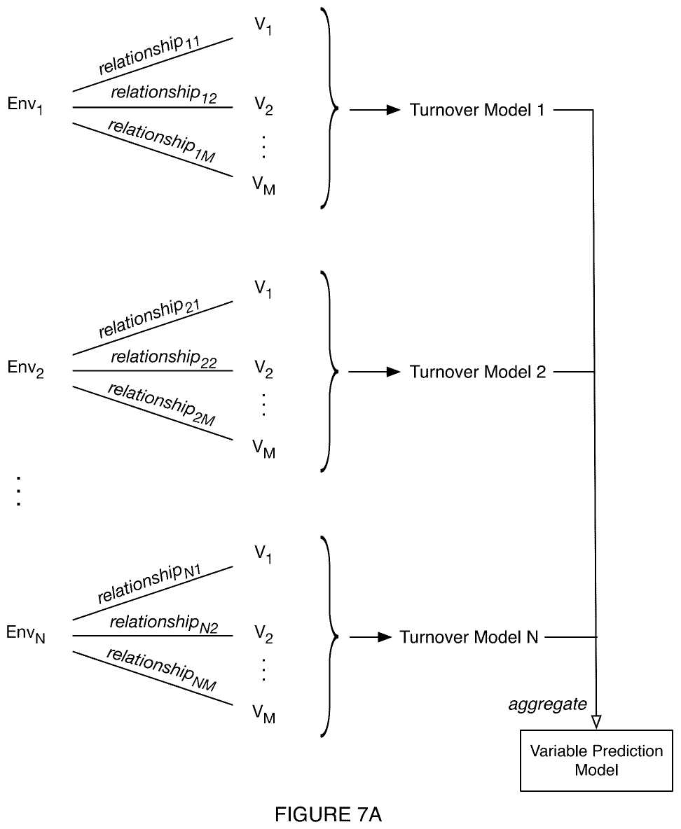

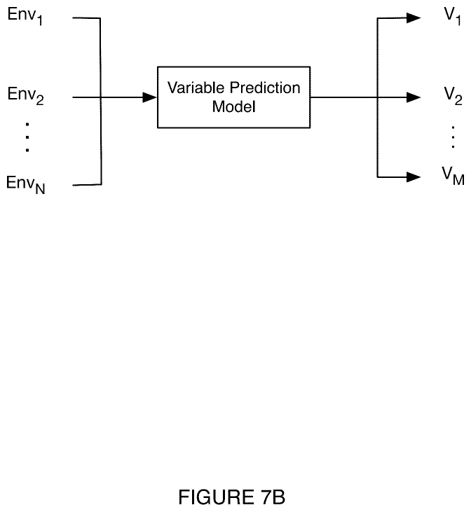

In a first variant, the variable prediction model can be or include a machine learning model. An example is shown in FIG. 7B. In a specific example, the variable prediction model can include a residual CNN with a multilayered perceptron (MLP) network. The variable prediction model can optionally have multiple heads (e.g., where a casual variable value is output at each head). In a second variant, the variable prediction model can be or include an aggregate of a set of models (e.g., turnover models), where each model in the set defines a relationship between an environmental parameter and a set of variables (e.g., the set of causal variables). In a specific example, each model in the set of models corresponding to an environmental parameter can be or include an aggregate of a set of submodels, where each submodel defines a relationship between the environmental parameter and a variable. An example is shown in FIG. 7A. In a specific example, the variable prediction model can include a gradient forest, random forest, and/or any other suitable model.

The variable prediction model can optionally be trained using observed environmental parameter values (e.g., one or more sets of observed environmental parameter values for each geographic region corresponding to a training organism) for a set of training organisms as the training input, and using observed variable values (e.g., genotypes, spectral features, etc.) for the set of training organisms as the training target. In a specific example, the set of training organisms can include a crop landrace, wherein the observed values (e.g., training values) for the set of environmental parameters can include environmental data corresponding to a geographic location of the crop landrace. The training data can be overlapping or nonoverlapping with training data used to determine the environment-variable association model. In an example, the environment-variable association model can be trained using training values (e.g., observed values) for a first set of environmental parameter values, and the variable prediction model can be trained using training values (e.g., observed values) for a second set of environmental parameter values. In an illustrative example, values for the first set of environmental parameters used to train the environment-variable association model do not include rasters, while values for the set of environmental parameters used to train the variable prediction model does include rasters. The variable prediction model can optionally be trained using a first set of training data (e.g., associated with a first species, associated with a first geographic region), then retrained (e.g., using transfer learning) using a second set of training data (e.g., associated with a second species, associated with a second geographic region, etc.). However, the variable prediction model can be otherwise trained.

However, the target set of causal variable values can be otherwise determined.

### 4.4. Evaluating an Organism Based on the Target Set of Causal Variable Values S400.

Evaluating an organism based on the target set of causal variable values S400 functions to predict fitness of the organism in a target environment (e.g., the target environment used in S300), to perform a risk analysis of exposing the organism to the target environment, to select an organism for development (e.g., for planting), to select an organism for breeding, and/or for any other organism analysis. S400 can optionally be performed for each organism in a set of organisms (e.g., a set of candidate organisms).

The organism can be any plant, animal, fungi, protist, moneran, and/or any other organism. In illustrative examples, the organism can be algae, broccoli, soy, sunflower, sugarcane, cotton, radishes, strawberry, dandelions, corn, bamboo, potatoes, mushrooms, herbs, pigs, cows, chickens, and/or any other organism. In specific examples, the organism can be used as food products, used to manufacture food products (e.g., as an ingredient in a food product), used to manufacture materials (e.g., rubber, oil, etc.), and/or used for any other purposes.

Evaluating the organism can include selecting the organism (e.g., for breeding, for planting, etc.), ranking the organism (within a set of candidate organisms), providing a score for the organism, and/or otherwise evaluating the set of candidate organisms. Examples of scores include: an offset metric, a breeding score, a fitness score (e.g., a projected chance of survival in the target environment), a risk score (e.g., a projected risk when exposed to the in the target environment), and/or any other metric.

Evaluating the organism can optionally include determining an observed set of causal variable values for the organism (e.g., observed values for the causal variables), and determining an offset metric based on the observed set of causal variable values and the target set of causal variable values (e.g., determined in S300). The observed set of causal variable values for the organism can be measured (e.g., a genomic sequence, a spectrum, etc.), extracted from measurements, predicted from measurements, estimated, and/or otherwise determined. For example, a set of sensors can be configured to receive data (e.g., genomic data, spectral data, etc.) for each of the set of candidate organisms, wherein the observed set of causal variable values (e.g., observed values for the causal variables) for each of the set of candidate organisms is determined based on the data. In an illustrative example, the observed set of causal variable values for the organism includes the genotype for the organism (for the causal genes).

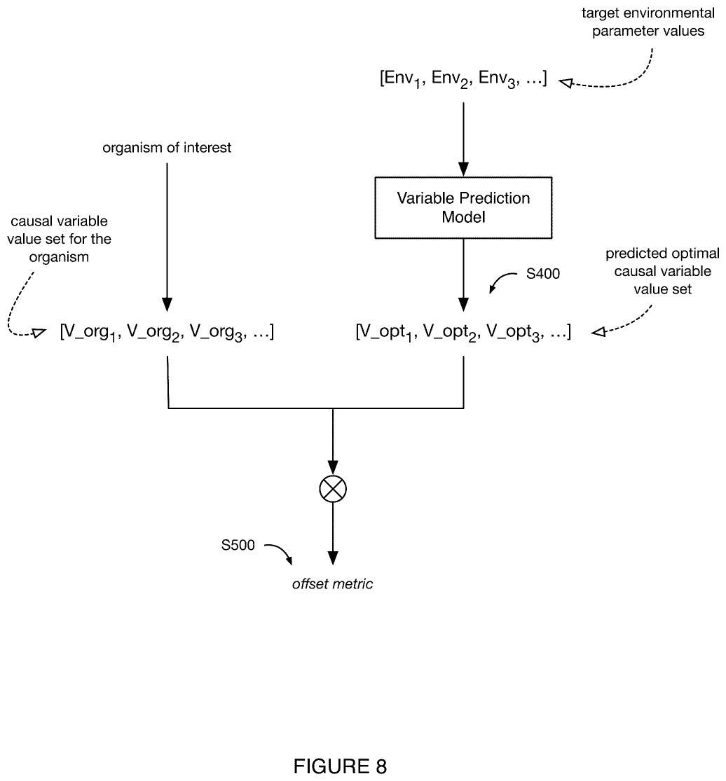

The offset metric can be determined using an offset model, determined using comparison methods, randomly determined, manually determined, and/or otherwise determined. The offset metric can be qualitative, quantitative, relative, discrete, continuous, a classification, numeric, binary, and/or be otherwise characterized. In an example, the offset metric can be or represent a measure of distance (e.g., genetic distance, vector distance, etc.) between the observed set of causal variable values and the target set of causal variable values. Variables can optionally be weighted (e.g., based on the respective association metric determined in S200). The offset metric can optionally be weighted based on a probability of occurrence of all or a portion of the observed set of causal variable values. However, any other comparison measure can be used. An example is shown in FIG. 8.

Evaluating the organism can optionally include selecting the organism from a set of candidate organisms for: further data collection (e.g., genomic sequencing), for breeding (e.g., cross-breeding), for breeding simulations, for development (e.g., the organism can be planted, grown, etc.), a combination thereof, and/or any other downstream use. The organism can be selected based on: the offset metric and/or any evaluation. Selecting the organism can include: not killing the organism (e.g., not weeding the organism), planting the organism, treating the organism (e.g., fertilizing the organism, replanting the organism, etc.), breeding (e.g., cross-breeding) the organism with one or more other organisms (e.g., a second selected organism, all other organisms in a selected set of organisms, etc.), and/or otherwise selecting the organism.

In an example, the candidate organisms can be ranked based on an evaluation metric, and candidate organisms are selected based on their ranking (e.g., the top 10% are selected, the top 100 organisms are selected, the organisms with an evaluation metric above a threshold value are selected, etc.). The evaluation metric for a candidate organism can optionally be the predicted characteristic value for the organism and/or be determined based on the predicted characteristic value. The evaluation metric can optionally be determined based on genetic variable values for the candidate organism and/or other candidate organisms (e.g., where increased genetic diversity of a candidate organism relative to the other candidate organisms increases the evaluation metric). In a specific example, organisms can be selected based on a simulation of how the selection process will optimize for a characteristic value over many generations (e.g., including accounting for genetic diversity). In a specific example, the subset of organisms can be selected based on an effective population size (e.g., determined based on genomic variable values).

In an illustrative example, selecting a subset of organisms includes: collecting spectral variable values for the set of candidate organisms; predicting characteristic values for the set of candidate organisms based on the spectral variable values (e.g., using the characteristic prediction model shown in FIG. 6A and/or FIG. 6B); selecting a subset of organisms from the set of candidate organisms based on the predicted characteristic values; determining genomic variable values for the subset of organisms; predicting updated characteristic values for the subset of organisms based on the genomic variable values and/or the spectral variable values; and selecting an updated subset of organisms from the subset of organisms based on the updated characteristic values.

However, the organism can be otherwise evaluated.

The method can optionally include determining breeding parameters, which functions to determine steps to reach the target set of causal variable values (e.g., to produce an organism optimized for a target environment) from an initial set of causal variable values. In a specific example, determining breeding parameters can include selecting one or more organisms for breeding based on the respective offset metrics.

The breeding parameters can include breeding sets (e.g., one or more organisms to cross-breed to achieve the target set of causal variable values), a number of breeding generations, treatments, growing conditions, and/or any other methods to transform an initial set of causal variable values to a target set of causal variable values. The breeding parameters preferably exclude genetic engineering (e.g., using CRISPR, foreign gene insertion, etc.), but can alternatively include genetic engineering.

In a first variant, the breeding parameters can be determined using predictive breeding methods. The predictive breeding methods can determine steps to breed one or more organisms—each associated with an initial set of causal variable values—to achieve a target organism associated with the target set of causal variable values. The one or more organisms can optionally be selected from a larger set of organisms (e.g., existing organisms currently available for breeding). The selected organisms can have the smallest offset metric (e.g., the closest causal variable values to the target causal variable values) and/or be otherwise selected. In an example, organism pairs can be selected (e.g., from a set of existing organisms, from a selected set of organisms, etc.) for breeding to achieve genotype values in a target set of causal variable value.

In a second variant, determining breeding parameters includes determining a treatment of an organism (e.g., applied at a given growth stage) that will alter one or more variable values to bring an initial set of causal variable values closer to the target set of causal variable values. The treatment can be determined using known effects of the treatment (e.g., known methylation effects), simulations of treatment at a growth stage, and/or using any other information associated with the treatment and/or the organism. The treatment values can be: predicted, manually specified, randomly determined, and/or otherwise determined. In a first example, a treatment of an organism can be determined to increase and/or decrease methylation of one or more genes (e.g., to alter causal DNA methylation variable values, to alter causal genomic expression variable values, etc.). In a second example, a gene therapy can be determined to increase and/or decrease gene expression for one or more genes (e.g., to alter causal genomic expression variable values). In a third example, genetic modification steps can be determined to modify an organism's genome (e.g., to alter causal variable values). Examples of treatments can include: irradiation, siRNA gene silencing, nutrient application, and/or other treatments. In a fourth example, the treatment values can be determined based on causal variable values (e.g., the genomic information of the organism being grown). In an illustrative example, a treatment amount and frequency (e.g., watering, fertilization, etc.) can be calculated given the variable values of the organism and the target variable values.

In an example, the breeding parameters can be determined using methods disclosed in U.S. application Ser. No. 18/884,930 filed 13 Sep. 2024, U.S. application Ser. No. 18/119,048 filed 8 Mar. 2023 and/or U.S. application Ser. No. 18/374,218 filed 28 Sep. 2023, each of which is incorporated in its entirety by this reference.

However, the breeding parameters can be otherwise determined.

### 5. Specific Examples

A numbered list of specific examples of the technology described herein are provided below. A person of skill in the art will recognize that the scope of the technology is not limited to and/or by these specific examples.

Specific Example 1. A method, comprising: determining a first model (e.g., environment-variable association model) defining a relationship between a set of genomic variables and a first set of environmental parameters; using the first model, determining an association metric for each genomic variable in the set of genomic variables; identifying causal genomic variables in the set of genomic variables based on the association metrics; training a second model (e.g., variable prediction model) using training data, wherein the training data comprises: for each of a set of training organisms, training values for the causal genomic variables and training values for a second set of environmental parameters; using the second model, predicting target values for the causal genomic variables based on target values for the second set of environmental parameters; and selecting an organism for breeding based the target values for the causal genomic variables.

Specific Example 2. The method of Specific Example 1, wherein selecting the organism for breeding comprises: collecting genomic data for the organism; determining observed values for the causal genomic variables based on the data; determining an offset metric for the organism based on a comparison between the target values for the causal genomic variables and the observed values for the causal genomic variables; and selecting the organism based on the offset metric.

Specific Example 3. The method of any of Specific Examples 1 or 2, wherein the training values for the second set of environmental parameters comprise a raster.

Specific Example 4. The method of any of Specific Examples 1-3, wherein the first model is determined using observed values for the first set of environmental parameters and observed values for the set of genomic variables, wherein the observed values for the first set of environmental parameters do not comprise a raster.

Specific Example 5. The method of any of Specific Examples 1-4, wherein determining an association metric for a genomic variable of interest in the set of genomic variables comprises: determining an observed metric based on an observed value for the genomic variable of interest; determining a test metric based on a test value for the genomic variable of interest, wherein the test value is determined based on values for a subset of genomic variables in the set of genomic variables, wherein the subset of genomic variables does not include the genomic variable of interest; and determining the association metric for the genomic variable of interest based on a comparison between the observed metric and the test metric.

Specific Example 6. The method of Specific Example 5, wherein the test value for the genomic variable of interest is determined using a third model (e.g., variable-variable association model), the third model defining a relationship between the genomic variable of interest and the subset of genomic variables.

Specific Example 7. The method of any of Specific Examples 1-6, wherein the target values for the second set of environmental parameters comprise values for a predicted future environment of a target geographical location.

Specific Example 8. The method of any of Specific Examples 1-7, wherein the target values for the second set of environmental parameters comprise measurements for a target geographical location.

Specific Example 9. The method of any of Specific Examples 1-8, wherein the set of training organisms comprises a crop landrace, wherein the training values for the second set of environmental parameters comprise environmental data corresponding to a geographic location of the crop landrace.

Specific Example 10. The method of any of Specific Examples 1-9, further comprising cross-breeding the selected organism with other organisms.

Specific Example 11. A system, comprising: a processing system configured to: select a set of causal variables associated with a first set of environmental parameters, wherein the set of causal variables is selected from a set of variables using an observed value and a test value for each variable in the set of variables, wherein a test value for a variable of interest in the set of variables is determined based on observed values for a subset of variables in the set of variables, wherein the subset of variables does not include the variable of interest; train a model to predict values for the set of causal variables based on values for a second set of environmental variables; using the model, predict target values for the set of causal variables based on target values for the second set of environmental variables; and select an organism from a set of candidate organisms for breeding based on: the target values for the set of causal variables and, for each of the set of candidate organisms, observed values for the causal variables.

Specific Example 12. The system of Specific Example 11, wherein selecting the organism comprises: determining an offset metric for the organism based on a comparison between the target values and the observed values for the organism; and selecting the organism based on the offset metric for the organism.

Specific Example 13. The system of any of Specific Examples 11 or 12, wherein selecting the set of causal variables associated with the first set of environmental parameters, comprises: training an environment-variable association model based on the observed values and the test values for the set of variables; using the trained environment-variable association model, determining an association metric for each variable in the set of variables; and selecting the set of causal variables based on the association metrics.

Specific Example 14. The system of any of Specific Examples 11-13, wherein the test value for the variable of interest is determined using a model trained to predict a value for the variable of interest based on values for the subset of variables.

Specific Example 15. The system of any of Specific Examples 11-14, wherein the target values for the second set of environmental parameters comprise a raster.

Specific Example 16. The system of any of Specific Examples 11-15, wherein the target values for the second set of environmental parameters comprise measurements for a target geographical location.

Specific Example 17. The system of any of Specific Examples 11-16, wherein the set of causal variables comprises genomic positions, wherein the target values for the set of causal variables comprise genotypes at the genomic positions.

Specific Example 18. The system of any of Specific Examples 11-17, further comprising a set of sensors configured to receive data for each of the set of candidate organisms, wherein the observed values for the causal variables for each of the set of candidate organisms are determined based on the data.

Specific Example 19. The system of Specific Example 18, wherein the set of sensors comprises image sensors, wherein the data comprises spectral data.

Specific Example 20. The system of Specific Example 18, wherein the data comprises genomic data.

As used herein, “substantially” or other words of approximation (e.g., “about,” “approximately,” etc.) can be within a predetermined error threshold or tolerance of a metric, component, or other reference (e.g., within +/−0.001%, +/−0.01%, +/−0.1%, +/−1%, +/−2%, +/−5%, +/−10%, +/−15%, +/−20%, +/−30%, any range or value therein, of a reference).

All references cited herein are incorporated by reference in their entirety, except to the extent that the incorporated material is inconsistent with the express disclosure herein, in which case the language in this disclosure controls.

Different subsystems and/or modules discussed above can be operated and controlled by the same or different entities. In the latter variants, different subsystems can communicate via: APIs (e.g., using API requests and responses, API keys, etc.), requests, and/or other communication channels. Communications between systems can be encrypted (e.g., using symmetric or asymmetric keys), signed, and/or otherwise authenticated or authorized.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A method, comprising:
determining a first model defining a relationship between a set of genomic variables and a first set of environmental parameters;
using the first model, determining an association metric for each genomic variable in the set of genomic variables;
identifying causal genomic variables in the set of genomic variables based on the association metrics;
training a second model using training data, wherein the training data comprises: for each of a set of training organisms, training values for the causal genomic variables and training values for a second set of environmental parameters;
using the second model, predicting target values for the causal genomic variables based on target values for the second set of environmental parameters; and
selecting an organism for breeding based the target values for the causal genomic variables.

2. The method of claim 1, wherein selecting the organism for breeding comprises:
collecting genomic data for the organism;
determining observed values for the causal genomic variables based on the data;
determining an offset metric for the organism based on a comparison between the target values for the causal genomic variables and the observed values for the causal genomic variables; and
selecting the organism based on the offset metric.

3. The method of claim 1, wherein the training values for the second set of environmental parameters comprise a raster.

4. The method of claim 3, wherein the first model is determined using observed values for the first set of environmental parameters and observed values for the set of genomic variables, wherein the observed values for the first set of environmental parameters do not comprise a raster.

5. The method of claim 1, wherein determining an association metric for a genomic variable of interest in the set of genomic variables comprises:
determining an observed metric based on an observed value for the genomic variable of interest;
determining a test metric based on a test value for the genomic variable of interest, wherein the test value is determined based on values for a subset of genomic variables in the set of genomic variables, wherein the subset of genomic variables does not include the genomic variable of interest; and
determining the association metric for the genomic variable of interest based on a comparison between the observed metric and the test metric.

6. The method of claim 5, wherein the test value for the genomic variable of interest is determined using a third model, the third model defining a relationship between the genomic variable of interest and the subset of genomic variables.

7. The method of claim 1, wherein the target values for the second set of environmental parameters comprise values for a predicted future environment of a target geographical location.

8. The method of claim 1, wherein the target values for the second set of environmental parameters comprise measurements for a target geographical location.

9. The method of claim 1, wherein the set of training organisms comprises a crop landrace, wherein the training values for the second set of environmental parameters comprise environmental data corresponding to a geographic location of the crop landrace.

10. The method of claim 1, further comprising cross-breeding the selected organism with other organisms.

11. A system, comprising:
a processing system configured to:
select a set of causal variables associated with a first set of environmental parameters, wherein the set of causal variables is selected from a set of variables using an observed value and a test value for each variable in the set of variables, wherein a test value for a variable of interest in the set of variables is determined based on observed values for a subset of variables in the set of variables, wherein the subset of variables does not include the variable of interest;
train a model to predict values for the set of causal variables based on values for a second set of environmental variables;
using the model, predict target values for the set of causal variables based on target values for the second set of environmental variables; and
select an organism from a set of candidate organisms for breeding based on: the target values for the set of causal variables and, for each of the set of candidate organisms, observed values for the causal variables.

12. The system of claim 11, wherein selecting the organism comprises:
determining an offset metric for the organism based on a comparison between the target values and the observed values for the organism; and
selecting the organism based on the offset metric for the organism.

13. The system of claim 11, wherein selecting the set of causal variables associated with the first set of environmental parameters, comprises:
training an environment-variable association model based on the observed values and the test values for the set of variables;
using the trained environment-variable association model, determining an association metric for each variable in the set of variables; and
selecting the set of causal variables based on the association metrics.

14. The system of claim 11, wherein the test value for the variable of interest is determined using a model trained to predict a value for the variable of interest based on values for the subset of variables.

15. The system of claim 11, wherein the target values for the second set of environmental parameters comprise a raster.

16. The system of claim 11, wherein the target values for the second set of environmental parameters comprise measurements for a target geographical location.

17. The system of claim 1, wherein the set of causal variables comprises genomic positions, wherein the target values for the set of causal variables comprise genotypes at the genomic positions.

18. The system of claim 11, further comprising a set of sensors configured to receive data for each of the set of candidate organisms, wherein the observed values for the causal variables for each of the set of candidate organisms are determined based on the data.

19. The system of claim 18, wherein the set of sensors comprises image sensors, wherein the data comprises spectral data.

20. The system of claim 18, wherein the data comprises genomic data.
